(19) United States
(12) Patent Application Publication
(10) Pub. No.: US 2026/0176594 A1
(43) Pub. Date: Jun. 25, 2026

(54) REDUCING DISEASE TRANSMISSION OF VECTOR BORNE DISEASES

(71) Applicant: GlaxoSmithKline Intellectual Property Development Limited, Stevenage (GB)

(72) Inventor: Janneth Fatima Indira RODRIGUES, Madrid (ES)

(73) Assignee: GlaxoSmithKline Intellectual Property Development Limited, Stevenage (GB)

(21) Appl. No.: 19/127,432

(22) PCT Filed: Nov. 8, 2023
(86) PCT No.: PCT/EP2023/081176

§ 371 Date: May 6, 2025

(30) Foreign Application Priority Data
Nov. 8, 2022 (EP) 22383076.1
Mar. 13, 2023 (EP) 23382233.7

(51) Int. Cl. C12N 7/00; A61K 9/00

(52) U.S. Cl. CPC C12N 7/00; A61K 9/0019

(57) ABSTRACT

The present application relates to compounds and pharmaceutically acceptable salts thereof, compositions thereof, and their use in the reduction or prevention of transmission of a parasite or vector borne disease.

### FIELD OF THE INVENTION

The present application relates to live bacteria and compounds and pharmaceutically acceptable salts thereof, compositions thereof, and their use in the reduction or prevention of transmission of a parasite or vector borne disease.

### BACKGROUND TO THE INVENTION

Infectious diseases are responsible for a wide variety of diseases of medical and veterinary importance. Many of these diseases are transmitted by insect vectors. Vector-borne diseases are infections transmitted by the bite of infected arthropod species, such as mosquitoes, ticks, triatomine bugs, sandflies, blackflies, as well as ectoparasites such as ticks and fleas.

Mosquitoes are vectors for a variety of infectious diseases. In particular, three medically relevant genus of mosquitoes which transmit diseases are Anopheles, Culex and Aedes. The genus Culex and Aedes belong to the sub-family Culicinae, while the Anopheles belongs to the sub-family Anophelinae. The Anopheles genus is a vector for malaria and filariasis. The Culex genus is a vector for Japanese encephalitis other viral diseases, and filariasis. The Aedes genus is a vector for dengue virus, chikungunya, Mayaro virus, Zika virus, yellow fever, Japanese encephalitis, West Nile, and filariasis.

Filariasis is a disease caused by roundworm parasites of the filarioidea type and is spread by blood-feeding insects such as black flies and mosquitoes. These are divided into categories, depending on where they affect. Lymphatic filariasis is caused by Wuchereria bancrofti, Brugia malayi, and Brugia timori, which occupy the lymphatic system and can lead to elephantiasis. Subcutaneous filariasis is caused by Loa (eye worm), Mansonella streptocerca, and Onchocerca volvulus, which occupy a layer just under the skin. Serious cavity filariasis is caused by the worms Mansonella perstans and Mansonella ozzardi, which occupy the serous cavity of the abdomen. Circulating microfilariae can be taken up during a blood meal by an insect vector; in the vector, they develop into infective larvae that can be spread to another person.

Zika virus disease is caused by a member of the virus family Flaviviridae. It is spread by daytime-active Aedes mosquitoes, such as Aedes aegypti and Aedes albopictus. Symptoms include fever, red eyes, joint pain, headache, and a maculopapular rash. Most cases have no symptoms, but when present they are usually mild and can resemble dengue fever, and generally last less than seven days. Infection during pregnancy may cause microcephaly and other brain malformations in babies.

West Nile virus is a single-stranded RNA virus that causes West Nile fever. It is a member of the Flaviviridae family, and is primarily transmitted by mosquitoes of the Culex species. About 80% of infected people have few or no symptoms. 20% of people infected develop a fever, headache, vomiting, or a rash. Less than 1% of people develop encephalitis or meningitis, with associated neck stiffness, confusion, or seizures, with the fatality rate being approximately 10%. Recovery may take weeks to months, with a 10% risk of death for those where the nervous system is affected.

Chikungunya virus is a member of the Alphavirus genus and Togaviridae family. It is an RNA virus with a positive sense single stranded genome of 11.7 kb. Chikungunya virus causes Chikungunya, which has symptoms of fever and joint pain generally 2-12 days after exposure. The risk of death is around 1 in 1,000. The disease causes an estimated 3 million infections each year. Chikungunya is present mostly in the developing world, but epidemics in the Indian Ocean, Pacific Islands, and in the Americas, continue to change the distribution of the disease.

Mayaro virus disease is caused by a member of the virus family Togaviridae, specifically the Alphavirus genus. Symptoms included fever, headache, myalgia, rash, prominent pain in the large joints, and association with rheumatic disease. It is known to circulate in South America. MAYV transmission is mainly maintained through the sylvatic cycle that involves non-human primates and Haemagogus mosquitoes.

Sand flies are vectors for leishmania. There are three main forms of Leishmaniasis: cutaneous leishmaniasis, mucocutaneous leishmaniasis, and visceral leishmaniasis. Cutaneous leishmaniasis is the most common form of leishmaniasis. Visceral leishmaniasis is the most serious form in which the parasites migrate to the vital organs. Visceral leishmaniasis is caused by the parasite Leishmania donovani, and is potentially fatal if untreated. Leishmaniasis circulates in the developing world, with about 90 percent of the world's cases of visceral leishmaniasis being in India, Bangladesh, Nepal, Sudan, and Brazil. Leishmaniasis affects 12 million people worldwide, with 1.5-2 million new cases each year. The visceral form of leishmaniasis has an estimated incidence of 500,000 new cases and 60,000 deaths each year. Kabul is estimated as the largest centre of cutaneous leishmaniasis in the world, with approximately 67,500 cases as of 2004.

Kissing bugs, also known as conenose bugs, are members of the Triatominae subfamily of reduviidae. These are vectors for Chagas disease, also known as American trypanosomiasis. Chagas disease is a parasite caused by the flagellate protozoa Trypanosoma cruzi. Chagas disease generally circulates in the Americas, and is endemic in poor, rural areas of Mexico, Central America, and South America. An estimated 10-15 million people are infected with Chagas disease a year, and about 14,000 people die annually. The symptoms of Chagas disease vary over the course of an infection. In the early (acute) stage, symptoms are mild, typically local swelling at the site of infection. After 4-8 weeks, individuals with active infections enter the chronic phase of Chagas disease that is asymptomatic for 60-80 percent of chronically infected individuals through their lifetime. However, the remaining 20-40 percent of infected people will develop debilitating and sometimes life-threatening medical problems over the course of their lives. Chagas disease is treated with nifurtimox and benznidazole, which cause significant side effects and are of negligible benefit in chronic disease.

Tsetse flies (Glossina spp.) are vectors for Human African Trypanosomiasis. Human African Trypanosomiasis, also called African Sleeping Sickness, is a parasitic disease caused by the protozoa Trypanosoma brucei. Two forms of the disease exist depending on the parasite sub-species. Trypanosoma brucei gambiense (T.B. gambiense) represents 95% of the reported cases and occurs in west and central Africa, causing chronic infection. Trypanosoma brucei rhodesiense (T.B. rhodesiense) is found in eastern and southern Africa, and represents about 5% of the cases.

These diseases are of significant medical importance. A number of drugs are available to treat and/or prevent some parasites or vector-borne diseases. However, not all parasites or vector-borne diseases can be treated efficiently. For example, there is currently no chemotherapeutic drug or vaccine available against the Dengue virus. Furthermore, in the case of antimalarial drugs, treatment with the drugs currently available is becoming less effective due to increased resistance in some Plasmodium strains. There is therefore the need to effectively control parasites and vectors of diseases to prevent transmission. In this regard, mosquitoes can be targeted by a wide range of insecticides and insect repellents. Mosquitoes can be targeted with insecticides when they are in a larval state or once they have developed into adults. However, mosquitoes have developed widespread resistance to currently used insecticides.

One approach to addressing this issue is to develop agents that are capable of reducing or preventing the transmission of vector borne diseases without negatively impacting the insect vector thereby circumventing the generation of resistance. In this regard, PCT/EP2020/069569 (published as WO 2021/009050) discloses bacteria of the Delftia genus, and its use in reducing malaria transmission in mosquitoes. Additionally, there is an existing need to develop novel modalities for reducing or preventing the transmission of vector borne diseases.

### SUMMARY OF THE INVENTION

According to a first aspect of the invention, there is provided a composition for use in a method of reducing or preventing transmission of a vector borne disease, wherein the composition comprises 1-Methyl-9H-pyrido[3,4-b]indole or a pharmaceutically acceptable salt thereof, and wherein the method comprises the step of bringing at least one vector into contact with the composition.

According to a second aspect of the invention, there is provided composition for use in a method of reducing or preventing transmission of a parasite, wherein the composition comprises 1-Methyl-9H-pyrido[3,4-b]indole or a pharmaceutically acceptable salt thereof, and wherein the method comprises the step of bringing at least one vector into contact with the composition.

In a third aspect of the invention, there is provided a method of reducing or preventing transmission of a vector borne disease or parasitic infection comprising a step of bringing at least one vector of the vector borne disease into contact with 1-Methyl-9H-pyrido[3,4-b]indole or a pharmaceutically acceptable salt thereof.

In a further aspect of the invention, there is provided the use of 1-Methyl-9H-pyrido[3,4-b]indole or a pharmaceutically acceptable salt thereof in reducing or preventing a vector borne disease or parasitic infection.

In a further aspect of the invention, there is provided 1-Methyl-9H-pyrido[3,4-b]indole or a pharmaceutically acceptable salt thereof for use in inhibiting the growth of a parasite.

In a further aspect of the invention, there is provided a nectar feed comprising 1-Methyl-9H-pyrido[3,4-b]indole or a pharmaceutically acceptable salt thereof, and one or more of a sugar source. Suitably, the nectar feed is for use in reducing or preventing a vector borne or parasitic disease.

In any of the above-listed aspects, the vector borne or parasitic disease may be selected from Human African Trypanosomiasis, Anaplasmosis, Babesiosis, Borrelia miyamotoi disease, Bourbon virus, Borrelia mayonii disease, Chikungunya virus, Chagas disease, Cryptosporidium. Dirofilariasis, Eastern Equine Encephalitis, Ehrlichia muris-like disease, Ehrlichiosis, Filariasis, Heartland virus, Japanese encephalitis, Leishmaniasis, Lyme Disease, Mayaro virus disease, Pacific Coast tick fever, Rocky Mountain spotted fever, St. Louis encephalitis, Tularemia, Western equine encephalitis, West Nile virus, Yellow fever, and Zika virus.

The present invention may be advantageous in a number of respects. In particular, the present inventors have found that the compound 1-Methyl-9H-pyrido[3,4-b]indole (also known as Harmane, or 1-methyl-β-carboline) is produced by bacteria of the Delftia genus that is responsible for hindering transmission of vector borne diseases and parasites in vectors (such as Mosquitoes, Sand flies, Tsetse flies, triatominae etc.). In some embodiments, the 1-Methyl-9H-pyrido[3,4-b]indole, Harmane, 1-methyl-β-carboline is produced by bacteria of the Delftia genus. When introduced into a vector containing environment, the compositions of the invention prevent parasite development in the vector and therefor interrupts disease transmission. 1-Methyl-9H-pyrido[3,4-b]indole can be used to combat the spread of parasites and vector borne diseases.

In a further aspect of the invention, there is provided a composition for use in a method of reducing or preventing transmission of a vector borne disease, wherein the composition comprises bacteria of the Delftia genus, and wherein the method comprises the step of bringing at least one vector into contact with the composition such that the vector orally ingests the composition, wherein the vector borne disease is selected from Human African Trypanosomiasis, Anaplasmosis, Babesiosis, Borrelia miyamotoi disease, Bourbon virus, Borrelia mayonii disease, Chikungunya virus, Chagas disease, Dirofilariasis, Eastern Equine Encephalitis, Ehrlichia muris-like disease, Ehrlichiosis, Filariasis, Heartland virus, Japanese encephalitis, Leishmaniasis, Lyme Disease, Mayaro virus disease, Pacific Coast tick fever, Rocky Mountain spotted fever, St. Louis encephalitis, Tularemia, Western equine encephalitis, West Nile virus, Yellow fever, and Zika virus.

In a further aspect of the invention, there is provided a composition for use in a method of reducing or preventing transmission of a parasite, wherein the composition comprises bacteria of the Delftia genus, and wherein the method comprises the step of bringing at least one vector into contact with the composition such that the vector orally ingests the composition, wherein the parasitic disease is selected from Human African Trypanosomiasis, Anaplasmosis, Babesiosis, Borrelia miyamotoi disease, Borrelia mayonii disease, Chagas disease, Cryptosporidium, Dirofilariasis, Ehrlichia muris-like disease, Ehrlichiosis, Filariasis, Leishmaniasis, Lyme Disease, Pacific Coast tick fever, Rocky Mountain spotted fever, and Tularemia. comprising bacteria of the Delftia genus.

In a further aspect of the invention, there is provided bacteria of the Delftia genus for use in reducing or preventing transmission of a disease selected from Human African Trypanosomiasis, Anaplasmosis, Babesiosis, Borrelia miyamotoi disease, Bourbon virus, Borrelia mayonii disease, Chikungunya virus, Chagas disease, Cryptosporidium. Dirofilariasis, Eastern Equine Encephalitis, Ehrlichia muris-like disease, Ehrlichiosis, Filariasis, Heartland virus, Japanese encephalitis, Leishmaniasis, Lyme Disease, Mayaro virus disease, Pacific Coast tick fever, Rocky Mountain spotted fever, St. Louis encephalitis, Tularemia, Western equine encephalitis, West Nile virus, Yellow fever, and Zika virus

Throughout this text, bacteria of the Delftia genus may suitably refer to Delftia tsuruhatensis TC1.

### BRIEF DESCRIPTION OF THE DRAWINGS

The invention is further described by reference to the accompanying drawings, which are non-limiting.

FIG. 1 shows a Liquid Chromatography-High Resolution accurate Mass Spectrometer trace for the active component of Delftia tsuruhatensis.

FIG. 2 shows a Heteronuclear single quantum coherence spectroscopy of the active component of Delftia tsuruhatensis.

FIG. 3 shows a 1H NMR spectrum of the active component of Delftia tsuruhatensis.

FIG. 4 shows the effect of Delftia tsuruhatensis on establishment of leishmania parasites in the sand fly midgut.

FIG. 5 shows the impact of timing of (i) feed and (ii) exposure to Delftia tsuruhatensis on establishment of leishmania parasites in the sand fly midgut.

FIG. 6 shows the impact of a second bloodmeal on L. major parasites in sandflies fed with Delftia tsuruhatensis.

FIG. 7 shows the effect of Delftia tsuruhatensis on sandflies infected naturally via bites to infected cutaneous lesions in mice.

FIG. 8 shows the effect on sandfly mortality of exposure to Delftia tsuruhatensis.

FIG. 9A shows the effect of other bacteria like E. coli or Ornithinibacillus massiliensis on establishment of leishmania parasites in the sand fly midgut.

FIG. 9B shows the effect of dead Delftia tsuruhatensis on establishment of leishmania parasites in the sand fly midgut.

FIG. 10 shows the effect of exposing sandflies to Delftia tsuruhatensis on the transmission of leishmania to mice.

FIG. 11 shows effect on Zika virus multiplication in mosquitoes which have ingested harmane.

### DETAILED DESCRIPTION OF THE EMBODIMENTS

In one aspect, the present invention provides a composition for use in a method of reducing or preventing transmission of a vector borne disease, wherein the composition comprises bacteria of the Delftia genus and/or 1-Methyl-9H-pyrido[3,4-b]indole or a pharmaceutically acceptable salt thereof, and wherein the method comprises the step of bringing at least one vector into contact with the composition.

In some embodiments, the composition comprises bacteria of the Delftia genus. In some embodiments, the composition comprises 1-Methyl-9H-pyrido[3,4-b]indole or a pharmaceutically acceptable salt thereof.

As used herein, the term “Vector-borne zoonotic disease” refers to a disease that naturally infects wildlife and is then transmitted to humans through carriers, or vectors, such as Mosquitoes, Ticks, Tsetse fly, black fly, Triatominae, and sand fly. As used herein, the term “Vector-borne disease” refers to a disease that naturally infects wildlife and is then transmitted to animals through carriers, or vectors, such as Mosquitoes, Ticks, Tsetse fly, black fly, Triatominae, and sand fly. In some embodiments, the animal is a human. In other embodiments, the animal is selected from the group consisting of dogs, cats, hamsters, cows, sheep, goats, pigs, rabbits, ducks, turkeys, horses, chickens, yaks, donkeys, buffalo, camels, or other domesticated animals.

Delftia is a genus of gram-negative motile rod bacteria, belonging to the class Betaproteobacteria and family Comamonadaceae.

The bacteria of the Delftia genus may be any bacteria in the Delftia genus. In an embodiment of the invention the bacteria of the Delftia genus is D. tsuruhatensis. Bacterium strain TC1 was deposited under the BUDAPEST TREATY ON THE INTERNATIONAL RECOGNITION OF THE DEPOSIT OF MICROORGANISMS FOR THE PURPOSES OF PATENT PROCEDURE at NCIMB Ltd (Ferguson Building, Craibstone Estate, Bucksburn, Aberdeen, AB21 9YA Scotland) on 21 May 2019, accession number NCIMB 43398. This bacterium has been isolated and identified by 16SrRNA sequencing as D. tsuruhatensis. It is a gram-negative bacterium belonging to the class Betaproteobacteria and the family Comamonadaceae. In an embodiment of the invention the bacteria of the Delftia genus is the bacteria deposited under the BUDAPEST TREATY ON THE INTERNATIONAL RECOGNITION OF THE DEPOSIT OF MICROORGANISMS FOR THE PURPOSES OF PATENT PROCEDURE (Ferguson Building, Craibstone Estate, Bucksburn, Aberdeen, AB21 9YA Scotland) on 21 May 2019, accession number NCIMB 43398 and the whole genome sequence has been deposited in NCBI (National Center for Biotechnological Information, National Library of Medicine, 8600 Rockville Pike, Bethesda, MD 20894, United States) on 14 Oct. 2022 with accession number PRJNA890603.

1-Methyl-9H-pyrido[3,4-b]indole is also known as harmane, and is represented by the following structure:

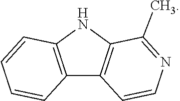 [SMILES: Cc1nccc2c1[nH]c1ccccc12]

This compound has been found to be the active compound secreted by Delftia bacteria, and is capable of suppressing parasite transmission in a variety of vectors. Thus, compositions of the present invention may reduce or prevent disease or parasitic transmission in a mosquito.

It will further be understood that the compounds of the invention, such as a compound of Formula (I) may exist in different tautomeric forms. Tautomers refer to isomeric forms of a compound that are in equilibrium with each other. The concentration of the isomeric forms will depend on the environment that the compound is in.

The compound may also be protonated or deprotonated depending on the pH of its surrounding environment. The compound may also be in the form of a pharmaceutically acceptable salt. Pharmaceutically acceptable salts include but are not limited to those described in Berge, J. Pharm. Sci., 1977, 66, 1-19, or those listed in P H Stahl and C G Wermuth, Handbook of Pharmaceutical Salts: Properties, Selection and Use, Second Edition, John Wiley & Sons, March 2011.

Where the compound functionality allows, suitable pharmaceutically acceptable salts of a compound of Formula (I) can be formed, which include acid or base addition salts. Acid addition salts may be formed by reaction with the appropriate acid, optionally in a suitable solvent such as an organic solvent, to give the salt which can be isolated by crystallisation and filtration. Base addition salts may be formed by reaction with the appropriate base, optionally in a suitable solvent such as an organic solvent, to give the salt which can be isolated by crystallisation and filtration.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′di (dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′dibenzylethylenediamine), bis-(2-hydroxyethyl) amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (/-methylglucamine), piperazine, piperidine, potassium, procaine, quinine, quinoline, sodium, strontium, f-butylamine, and zinc.

The compound will be administered in the appropriate “effective amount”. This effective amount will depend upon a number of factors including, for example, the size and weight of the subject, the precise condition requiring treatment and its severity, the nature of the formulation, and the route of distribution, and will ultimately be at the discretion of the skilled person.

In some embodiments, the vector borne disease is selected from African Trypanosomiasis, Anaplasmosis, Babesiosis, Borrelia miyamotoi disease, Bourbon virus, Borrelia mayonii disease, Chikungunya virus, Chagas disease, Dirofilariasis, Eastern Equine Encephalitis, Ehrlichia muris-like infection, Ehrlichiosis, Filariasis, Heartland virus, Japanese encephalitis, Leishmaniasis, Lyme Disease, Mayaro virus disease, Pacific Coast tick fever, Rocky Mountain spotted fever, St. Louis encephalitis, Tularemia, Western equine encephalitis, West Nile virus, Yellow fever, and Zika virus.

In some embodiments, the composition comprises bacteria of the Delftia genus and the vector borne disease is selected from African Trypanosomiasis, Anaplasmosis, Babesiosis, Borrelia miyamotoi disease, Bourbon virus, Borrelia mayonii disease, Chikungunya virus, Chagas disease, Dirofilariasis, Eastern Equine Encephalitis, Ehrlichia muris-like infection, Ehrlichiosis, Filariasis, Heartland virus, Japanese encephalitis, Leishmaniasis, Lyme Disease, Mayaro virus disease, Pacific Coast tick fever, Rocky Mountain spotted fever, St. Louis encephalitis, Tularemia, Western equine encephalitis, West Nile virus, Yellow fever, and Zika virus.

In some embodiments, the composition comprises 1-Methyl-9H-pyrido[3,4-b]indole or a pharmaceutically acceptable salt thereof and the vector borne disease is selected from African Trypanosomiasis, Anaplasmosis, Babesiosis, Borrelia miyamotoi disease, Bourbon virus, Borrelia mayonii disease, Chikungunya virus, Chagas disease, Dirofilariasis, Eastern Equine Encephalitis, Ehrlichia muris-like infection, Ehrlichiosis, Filariasis, Heartland virus, Japanese encephalitis, Lyme Disease, Mayaro virus disease, Pacific Coast tick fever, Rocky Mountain spotted fever, St. Louis encephalitis, Tularemia, Western equine encephalitis, West Nile virus, Yellow fever, and Zika virus.

In a second aspect, the present invention provides a composition for use in a method of reducing or preventing transmission of a parasite, wherein the composition comprises bacteria of the Delftia genus and/or 1-Methyl-9H-pyrido[3,4-b]indole or a pharmaceutically acceptable salt thereof, and wherein the method comprises the step of bringing at least one vector into contact with the composition.

In some embodiments, the parasitic disease is selected from Human African Trypanosomiasis, Anaplasmosis, Babesiosis, Borrelia miyamotoi disease, Borrelia mayonii disease, Chagas disease, Cryptosporidium, Dirofilariasis, Ehrlichia muris-like infection, Ehrlichiosis, Filariasis, Leishmaniasis, Lyme Disease, Pacific Coast tick fever, Rocky Mountain spotted fever, and Tularemia.

In some embodiments, the composition for use in a method of reducing or preventing transmission of a parasite comprises bacteria of the Delftia genus and the parasitic disease is selected from Human African Trypanosomiasis, Anaplasmosis, Babesiosis, Borrelia miyamotoi disease, Borrelia mayonii disease, Chagas disease, Cryptosporidium, Dirofilariasis, Ehrlichia muris-like infection, Ehrlichiosis, Filariasis, Leishmaniasis, Lyme Disease, Pacific Coast tick fever, Rocky Mountain spotted fever, and Tularemia.

In some embodiments, the composition for use in a method of reducing or preventing transmission of a parasite comprises 1-Methyl-9H-pyrido[3,4-b]indole or a pharmaceutically acceptable salt thereof and the parasitic disease is selected from Human African Trypanosomiasis, Anaplasmosis, Babesiosis, Borrelia miyamotoi disease, Borrelia mayonii disease, Chagas disease, Cryptosporidium, Dirofilariasis, Ehrlichia muris-like infection, Ehrlichiosis, Filariasis, Lyme Disease, Pacific Coast tick fever, Rocky Mountain spotted fever, and Tularemia.

Specifically, it has been shown here that, when introduced into a vector containing environment, harmane can prevent parasite transmission. In some embodiments, the mode of introduction to the vector can be through contact with sugar baits, nectar baits, blood baits, and/or other feeding baits where bacteria of the Delftia genus and/or the 1-Methyl-9H-pyrido[3,4-b]indole can be transmitted to and/or into the vector through cuticular uptake and/or ingestion. Thus, compositions of the present invention may reduce or prevent disease transmission and/or parasite transmission in a vector. The vector may be any vector capable of transmitting disease, and may in some embodiments be a mosquito e.g. mosquitoes of the Anopheles genus. It is envisaged that the compositions and methods of the present invention extend to any Anopheles species of mosquito. In an embodiment, the mosquito is Anopholes gambiae, Anopholes stephensi, Anopholes culicifacies, or Anopholes coluzzi. In an embodiment of the invention the mosquito is Anopheles gambiae or Anopheles stephensi. In an embodiment the mosquito is Anopheles stephensi. In another embodiment, the mosquito is Anopheles gambiae.

In some embodiments, the composition is for use in reducing or preventing: (i) Zika virus, and/or (ii) Zika virus transmission. In some embodiments, the composition is for use in reducing or preventing: (i) Zika virus, and/or (ii) Zika virus transmission in a mosquito. In some embodiments, the composition is for use in reducing or preventing Zika virus. In some embodiments, the composition is for use in reducing or preventing Zika virus transmission in a mosquito. The mosquito may be any mosquito. In some embodiments, the mosquito is a mosquito of the Aedes genus. In some embodiments, the mosquito is Aedes albopictus, or Aedes aegypti.

In some embodiments, the composition is for use in reducing or preventing: (i) Chikungunya virus, and/or (ii) Chikungunya virus transmission. In some embodiments, the composition is for use in reducing or preventing: (i) Chikungunya virus, and/or (ii) Chikungunya virus transmission in a mosquito. In some embodiments, the composition is for use in reducing or preventing Chikungunya virus. In some embodiments, the composition is for use in reducing or preventing Chikungunya virus transmission in a mosquito. The mosquito may be any mosquito. In some embodiments, the mosquito is a mosquito of the Aedes genus. In some embodiments, the mosquito is Aedes albopictus, or Aedes aegypti.

In some embodiments, the composition is for use in reducing or preventing: (i) Filariasis or Dirofilariasis, and/or (ii) Filariasis or Dirofilariasis transmission. In some embodiments, the composition is for use in reducing or preventing: (i) Filariasis or Dirofilariasis, and/or (ii) Filariasis or Dirofilariasis transmission in a mosquito. In some embodiments, the composition is for use in reducing or preventing Filariasis or Dirofilariasis. In some embodiments, the composition is for use in reducing or preventing Filariasis or Dirofilariasis transmission in a mosquito. The mosquito may be any mosquito. In some embodiments, the mosquito is a mosquito of the Aedes genus. In some embodiments, the mosquito is Aedes albopictus, or Aedes aegypti.

In some embodiments, the composition is for use in reducing or preventing: (i) Mayaro virus, and/or (ii) Mayaro virus transmission. In some embodiments, the composition is for use in reducing or preventing: (i) Mayaro virus, and/or (ii) Mayaro virus transmission in a mosquito. In some embodiments, the composition is for use in reducing or preventing Mayaro virus. In some embodiments, the composition is for use in reducing or preventing Mayaro virus transmission in a mosquito. The mosquito may be any mosquito. In some embodiments, the mosquito is a mosquito of the Aedes genus. In some embodiments, the mosquito is Aedes albopictus, or Aedes aegypti.

In some embodiments, the composition is for use in reducing or preventing: (i) West Nile virus, and/or (ii) West Nile virus transmission. In some embodiments, the composition is for use in reducing or preventing: (i) West Nile virus, and/or (ii) West Nile virus transmission in a mosquito. In some embodiments, the composition is for use in reducing or preventing West Nile Virus transmission in a mosquito. The mosquito may be any mosquito. In some embodiments, the mosquito is a mosquito of the Aedes genus. In some embodiments, the mosquito is Aedes albopictus, or Aedes aegypti.

In some embodiments, the composition is for use in reducing or preventing: (i) Chagas disease, and/or (ii) Chagas disease transmission. In some embodiments, the composition is for use in reducing or preventing Chagas disease transmission in a Triatominae. In some embodiments, the composition is for use in reducing or preventing Chagas disease parasite transmission in a Triatominae. In some embodiments, the Triatominae is Triatoma dimidiate.

In some embodiments, the composition is for use in reducing or preventing: (i) Human African Trypanosomiasis, and/or (ii) Human African Trypanosomiasis transmission. In some embodiments, the composition is for use in reducing or preventing: (i) Human African Trypanosomiasis, and/or (ii) Human African Trypanosomiasis transmission in a Tsetse fly. In some embodiments, the composition is for use in reducing or preventing Human African Trypanosomiasis. In some embodiments, the composition is for use in reducing or preventing Human African Trypanosomiasis transmission in a Tsetse fly. In some embodiments, the Tsetse fly is of the Glossina genus. In some embodiments, the Tsetse fly is Glossina palpalis.

In some embodiments, the composition is for use in reducing or preventing: (i) Leishmaniasis, and/or (ii) Leishmaniasis transmission. In some embodiments, the composition is for use in reducing or preventing: (i) Leishmaniasis, and/or (ii) Leishmaniasis transmission in a Sand fly. In some embodiments, the composition is for use in reducing or preventing Leishmaniasis. In some embodiments, the composition is for use in reducing or preventing Leishmaniasis transmission in a Sand fly. In some embodiments, the Sand fly is of the genus Phlebotomus or Lutzomyia.

In some embodiments, the composition comprises bacteria of the Delftia genus and is for use in reducing or preventing: (i) Leishmaniasis, and/or (ii) Leishmaniasis transmission. In some embodiments, the composition is for use in reducing or preventing: (i) Leishmaniasis, and/or (ii) Leishmaniasis transmission in a Sand fly. In some embodiments, the composition is for use in reducing or preventing Leishmaniasis. In some embodiments, the composition is for use in reducing or preventing Leishmaniasis transmission in a Sand fly. In some embodiments, the Sand fly is of the genus Phlebotomus or Lutzomyia.

In some embodiments, the composition is for use in reducing or preventing: (i) Cryptosporidium, and/or (ii) Cryptosporidium transmission. In some embodiments, the composition is for use in reducing or preventing Cryptosporidium. In some embodiments, the composition is for use in reducing or preventing Cryptosporidium transmission.

In another aspect, the present invention provides a method of reducing or preventing transmission of a vector borne disease or parasite comprising a step of bringing at least one vector of the vector borne disease or parasite into contact with bacteria of the Delftia genus and/or 1-Methyl-9H-pyrido[3,4-b]indole or a pharmaceutically acceptable salt thereof. The vector borne disease or parasite may be as defined above.

The step of bringing the vector or parasite into contact with the composition may occur in any suitable way. For instance, a person does not physically have to contact a vector or parasite with bacteria of the Delftia genus and/or 1-Methyl-9H-pyrido[3,4-b]indole or a pharmaceutically acceptable salt thereof. The composition could be left in a place where it will come into contact with the vector. The composition may be in a form as described above or below.

In certain embodiments of the invention, the contacting may be achieved by treating an area with a composition of the present invention, for example, by using a spray formulation, such as an aerosol or a pump spray. In certain embodiments of the invention, an area can be treated, for example, via aerial delivery, by truck-mounted equipment, or the like. In some embodiments, the composite on is sprayed by e.g., backpack spraying, aerial spraying, spraying/dusting etc. The vector or parasite may be any vector of parasite capable of transmitting disease. In some embodiments, the vector is a mosquito, black fly, triatominae, sand fly, or tsetse fly.

It is envisaged that the compositions and methods of the present invention extend to any species of mosquito. In some embodiments, the mosquito is a mosquito of the Anopholes genus. In some embodiments, the mosquito is Anopholes gambiae, Anopholes stephensi, Anopholes culicifacies, or Anopholes coluzzi. In some embodiments, the mosquito is Anopheles stephensi or Anopheles gambiae. In an embodiment the mosquito is Anopheles stephensi. In another embodiment, the mosquito is Anopheles gambiae.

For Filariasis, the method involves contacting a mosquito or black fly with-Methyl-9H-pyrido[3,4-b]indole or a pharmaceutically acceptable salt thereof. The mosque may be any mosquito or black fly capable of transmitting Filariasis. It is envisaged that the compositions and methods of the present invention extend to any species of mosquito or black fly.

In some embodiments, the dirofilariasis is caused by Dirofilaria immitis, Dirofilaria repens or Dirofilaria tenuis. In some embodiments, the dirofilariasis is caused by Dirofilaria immitis. In some embodiments, the dirofilariasis is caused by Dirofilaria repens. In some embodiments, the dirofilariasis is caused by Dirofilaria tenuis.

In some embodiments, the Filariasis is caused by worms of the Filarioidea genus. In some embodiments, the worm is Wuchereria bancrofti, Brugia malayi, or Brugia timori. In some embodiments, the worm is Loa loa, Mansonella streptocerca, or Onchocerca volvulus. In some embodiments, the worm is Mansonella perstans or Mansonella ozzardi.

For Chagas disease, the method involves contacting an infected Triatominae with the composition of the invention. The Triatominae may be any Triatominae capable of transmitting Chagas disease, e.g. Triatoma infestans, Triatoma dimidiate, or Rhodnius prolixus. In some embodiments, the Triatominae is Triatoma infestans. In some embodiments, the Triatominae is Triatoma dimidiate. In some embodiments, the Triatominae is Rhodnius prolixus. The parasite may be any parasite that causes Chagas disease. In some embodiments, the parasite is Trypanosoma cruzi.

For Leishmaniasis, the method involves contacting an infected sand fly with the composition of the invention. In some embodiments, the method involves contacting an infected sand fly with a composition comprising bacteria of the Delftia genus. The sand fly may be any sand fly capable of transmitting Leishmania. It is believed that the compositions and methods of the present invention extend to any species of sand fly. In some embodiments, the sand fly is of the genus Phlebotomus or Lutzomyia.

The parasite may be any Leishmaniasis parasite. In some embodiments, the parasite is Leishmania braziliensis, Leishmania donovani, Leishmania infantum, Leishmania chagasi, Leishmania panamensis, Leishmania guayanensis, Leishmania amazonensis, Leishmania mexicana, Leishmania tropica, or Leishmania major. In some embodiments, the parasite is Leishmania donovani. In some embodiments, the parasite is Leishmania infantum. In some embodiments, the disease being treated is visceral leishmaniasis. In another embodiment, the disease being treated is cutaneous leishmaniasis.

For Human African Trypanosomiasis, the method involves contacting an infected Tsetse fly with the composition of the invention. The Tsetse fly may be any Tsetse fly capable of transmitting Human African Trypanosomiasis. It is believed that the compositions and methods of the present invention extend to any species of Tsetse fly. In some embodiments, the Tsetse fly is Glossina palpalis.

The parasite may be any parasite that causes Human African Trypanosomiasis. In some embodiments, the parasite is selected from Trypanosoma brucei gambiense (TbG) and Trypanosoma brucei rhodesiense (TbR). In some embodiments, the parasite is selected from Trypanosoma brucei gambiense (TbG). In some embodiments, the parasite is Trypanosoma brucei rhodesiense (TbR).

For Cryptosporidium, the method involves contacting Cryptosporidium with the composition of the invention. Cryptosporidium is an apicomplexan parasitic alveolate that causes respiratory and gastrointestinal illness. The parasite may be any parasite that causes Cryptosporidium, and it is believed that the compositions and methods of the present invention extend to any Cryptosporidium parasite. In some embodiments, the parasite is selected from Cryptosporidium parvum, Cryptosporidium hominis, Cryptosporidium canis, Cryptosporidium felis, Cryptosporidium meleagridid and Cryptosporidium muris.

In another aspect, the present invention provides the use of bacteria of the Delftia genus and/or 1-Methyl-9H-pyrido[3,4-b]indole or a pharmaceutically acceptable salt thereof in reducing or preventing a vector borne disease or parasitic infection.

### Compositions

The compositions of the invention may be in any suitable form and may include any suitable carrier. The composition may be a feed composition, i.e. the composition may be in a form which can be presented to a vector for consumption through oral administration. In an embodiment the feed composition is a sugar source or nectar feed. In some embodiments, the feed composition is a sugar source. The sugar source may be an attractive sugar bait or be comprised within an attractive sugar bait. Attractive sugar baits comprise a sugar and a toxic ingredient. It is envisaged that an attractive sugar bait according to the invention will comprise the composition of the invention instead of the toxic ingredient, i.e. will comprise sugar and a composition of the invention. In an embodiment, the composition is in the form of a bait. The bait is designed to lure the vector (e.g., mosquito) to come into contact with the composition. In some embodiments, upon coming into contact therewith, the composition is then internalized by the vector or parasite (e.g., mosquito), by ingestion for example. An attractant can also be used. The attractant can be a pheromone, such as a male or female pheromone. The attractant acts to lure the vector (e.g., mosquito) to the bait. The bait can be in any suitable form, such as a solid, paste, pellet or powdered form.

The baits can be provided in a suitable “housing” or “trap”. Such housings and traps are commercially available and existing traps can be adapted to include the compositions of the invention. The housing or trap can, for example, be box-shaped and can be provided in pre-formed condition or can be formed of foldable cardboard for example. Suitable materials for a housing or trap include plastics and cardboard, particularly corrugated cardboard. The inside surfaces of the traps can be lined with a sticky substance in order to restrict movement of the vector or parasite (e.g., mosquito) once inside the trap. The housing or trap can contain a suitable trough inside which can hold the bait in place. A trap is distinguished from a housing because the mosquito cannot readily leave a trap following entry, whereas a housing acts as a “feeding station” which provides the vector (e.g., mosquito) with a preferred environment in which they can feed and feel safe from predators.

In another embodiment, the present invention provides a mosquito nectar feed comprising bacteria of the Delftia genus and/or 1-Methyl-9H-pyrido[3,4-b]indole or a pharmaceutically acceptable salt thereof, and one or more of a sugar source.

In a further aspect of the invention, bacteria of the Delftia genus can be used in a disease control strategy based on the direct exposure of larval or adult stage vectors to the bacterium. Exposure could be achieved through direction administration of the bacteria.

Alternatively, the bacteria can be deployed directly to populate a local vector population.

The bacteria may be delivered by any suitable method and in combination with a delivery agent in any suitable manner that permits administering the composition to the vector. For example, the vector can be contacted with the bacteria in a pure or substantially pure form, for example a solution containing Delftia.

In a particular embodiment, the bacteria of the Delftia genus is in a composition along with a delivery agent.

In another particular embodiment, the vector (e.g. mosquito) larval forms can be simply “soaked” or “sprayed” with a solution comprising the bacteria.

### Combinations

It is envisaged that the present invention is deployed along with other anti-vector or anti-parasitic eradication efforts. For example, the compositions, methods and compound for use of the present invention may be used alongside known anti-vector or anti-parasite agents (such as anti-malarial agents). In some embodiments, the compositions or compound for use of the present invention may be used in combination with one, two or three additional anti-parasite agent (e.g., anti-malarial agents). Integrated Vector Management (IVM) suggests making full use of the tools available.

The at least one other anti-malarial agent may also be selected from ferroquine, KAF156, cipargamin, DSM265, artemisone, artemisinone, artefenomel, MMV048, SJ733, P218, MMV253, PA92, DDD498, AN13762, DSM421, UCT947, ACT 451840, OZ609, OZ277 and SAR97276. In the treatment of P. falciparum infections, the at least one, two or three additional anti-malarial agents may be selected from the following list, wherein at least one of the anti-malarial agents is an artemisinin-based agent: artemether and lumefantrine, artesunate and amodiaquine, artesunate and mefloquine, dihydroartemisinin and piperaquine, or artesunate and sulfadoxine-pyrimethamine (SP). The above combination treatments are known as artemisinin-based combination therapies (ACTs). The choice of ACT is usually based on the results of therapeutic efficacy studies against local strains of P. falciparum malaria. In the treatment of P. vivax infections, an ACT may be used, as described above. Alternatively, the at least one other anti-malarial agent may be chloroquine, particularly in areas without chloroquine resistant P. vivax. In areas where resistant P. vivax has been identified, infections may be treated with an ACT, as described above. The combinations of therapeutic agents may conveniently be presented for use in the form of a pharmaceutical composition or formulation and may be administered together or separately and, when administered separately, this may occur separately or sequentially in any order (by the same or by different routes of administration).

The compositions or bacteria for use of the invention may be used in conjunction with use insecticide treated nets (ITNs), including long-acting insecticidal nets (LLINs) and/IRS (Indoor residual sprays). ATSB (attractive toxic sugar baits) lure mosquitos to feed on sugar with toxic mosquito-killing compounds. For efficiency, ATSB's could also include the harmane compound discussed above, instead of the toxic compound.

### EXAMPLES

The invention will now be illustrated by way of the following non-limiting examples. While particular embodiments of the invention are described below a skilled artisan will appreciate that various changes and modifications can be made. References to preparations carried out in a similar manner to, or by the general method of, other preparations, may encompass variations in routine parameters such as time, temperature, work-up conditions, and minor changes in reagent amounts, etc.

### Supernatant Preparation

D. tsuruhatensis TC1 was grown in LB liquid medium overnight (200 rpm, 28° C.). Bacteria were washed, resuspended in M9 medium (109/ml) and incubated (200 rpm, 28° C.) for 8 h, after which they were centrifuged, and the supernatant passed through a 0.22 μm filter to generate D. tsuruhatensis TC1 (D-8h). D. tsuruhatensis TC1 supernatant was passed through a 3 kDa centrifugal filter (Amicon Ultra-3K, REF: UFC500396) to generate a <3 KDa fraction.

### Example 1: Bioassay Guided Purification of the Active Natural Component from the Supernatant of Delftia tsuruhatensis TC1

The fermentation supernatant (10 L) was loaded onto a C-18 reversed-phase silica gel column (160×30 mm; Sepra™ C-18-E (50 μm, 65 Å)) for flash fractionation.

Unretained material in the column (follow-through was collected for activity testing) and the column was eluted subjected to isocratic elution (H₂O/CH₃CN 95:5) followed by a gradient of acetonitrile (CH₃CN) in water from 5% to 100% in 40 min, and an isocratic step at 100% of CH₃CN for 20 min at 10 mL/min. 18 mL fractions were collected. UV detection at 210 and 280 nm was used.

Additionally, 100 ml of unfermented medium were loaded onto a 60×15 mm C-18 cartridge and eluted with 100 ml of 100% of CH₃CN, that were dried down (12.5 mg) and were dissolved in MeOH 1 mL.

500 μL of the supernatant, aliquots of 500 μL of each fraction, 500 μL of the follow-through, obtained while loading the 10 L supernatant in the C-18 column, and 100 μL of the blank medium extract were transferred into an AB-Gene 0765 800 μL 96-well storage plate and dried down in a HT-8 Genevac vacuum centrifuge for shipment and activity evaluation.

### LC-HRMS Dereplication Methodology

Active fractions were analyzed using an Agilent 1200 Rapid Resolution HPLC interfaced to a Bruker maXis mass spectrometer. The volume of sample injected was 2 μL. A Zorbax SB-C8 column (2.1×30 mm, 3.5 μm particle size) was used for the separation. Two solvents were used as mobile phase: solvent A H₂O:CH₃CN 90:10, solvent B water: CH₃CN 10:90, both with 13 mM ammonium formate and 0.01% TFA. The gradient composition was:

| Time (minutes) | A | B | Flow (mL/min) |
|---|---|---|---|
| 0 | 90 | 10 | 0.3 |
| 6 | 0 | 100 | 0.3 |
| 8 | 0 | 100 | 0.3 |
| 8.1 | 90 | 10 | 0.3 |
| 10 | 90 | 10 | 0.3 |

The mass spectrometer was operated in positive ESI mode. The instrumental parameters were: 4 kV capillary voltage, drying gas flow of 11 L/min at 200° C., nebulizer pressure at 2.8 bars. TFA-Na cluster ions were used for mass calibration of the instrument prior to samples injection. Each sample run was recalibrated by infusion with the same TFA-NA calibrant before the chromatographic front.

### NMR Dereplication Methodology

For the NMR analyses, samples were dissolved in CD₃OD. After dissolution, each sample was transferred to a 1.7 mm tube. Acquisitions (1D 1H spectra and 2D HSQC spectra) were carried out on a Bruker AVANCE III 500 MHz spectrometer equipped with a 1.7 mm TCI micro-cryoprobe. All spectra were registered at 24° C.

### Results

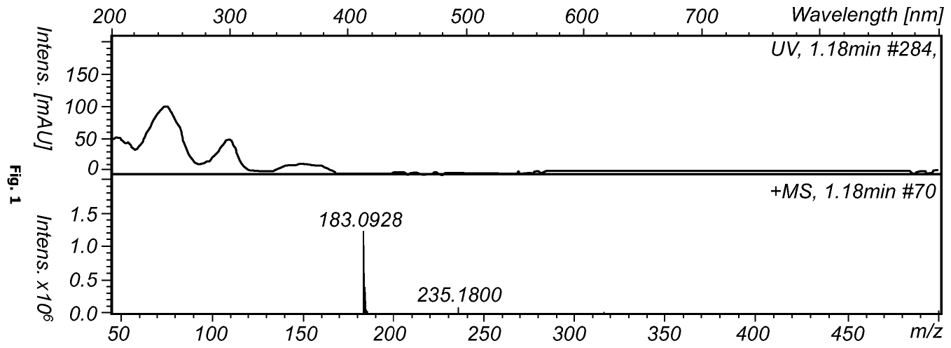

A molecular formula of C₁₂H₁₀N₂ was determined for the active component from LC-HRMS as shown in FIG. 1.

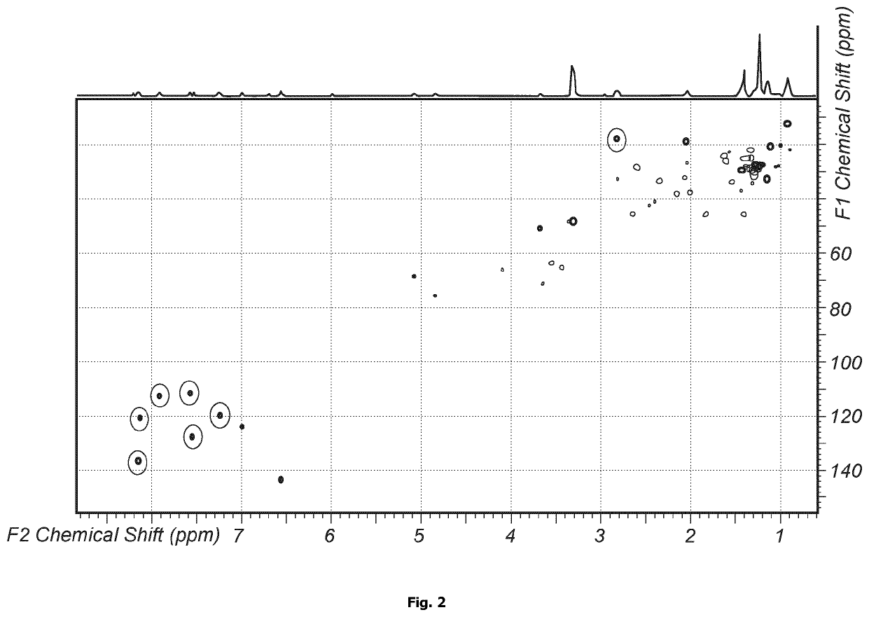

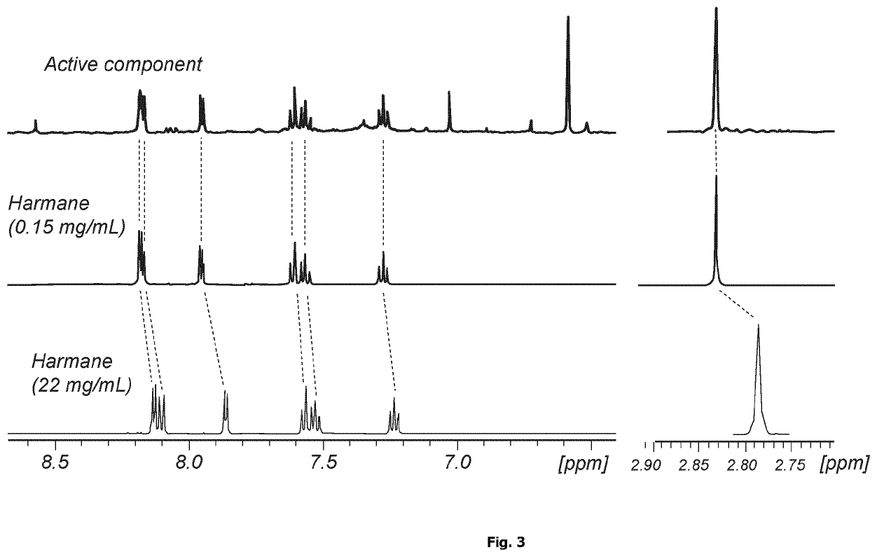

The HSQC and ¹H NMR spectra for the active component matched that of harmane as shown in FIGS. 2 and 3.

### Example 2: Effect of Delftia tsuruhatensis on Establishment of Leishmania Parasites in Sandfly Midgut

### Materials & Methods Used to Establish the Role of Delftia TC1 in Interruption of Leishmania Infection in the Sand Fly

### Parasites

A cloned line of Leishmania major (WR 2885) was used [Cecílio P, Pires ACAM, Valenzuela J G, et al. Exploring Lutzomyia longipalpis sand fly vector competence for Leishmania major parasites, The Journal of Infectious Diseases. 2020. 222 (7): 1199-1203. https://doi.org/10.1093/infdis/jiaa203.].

Promastigotes were maintained at 26° C. in Schneider's insect medium supplemented with 10% heat-inactivated fetal bovine serum, 100 U/mL penicillin, and 100 mg/ml streptomycin (all Thermo Fischer Scientific).

### Mice

Six-week-old female BALB/c mice were obtained from Charles River laboratories and housed under pathogen-free conditions at the NIAID Twinbrook animal facility (Rockville, MD) with water and food ad libitum.

### Sand Flies

Phlebotomus duboscqui sand flies were mass reared at the Laboratory of Malaria and Vector Research insectary as described by Lawyer P, Killick-Kendrick M, Rowland T, et al. Laboratory colonization and mass rearing of phlebotomine sand flies (Diptera, Psychodidae). Parasite. 2017. 24:42.

Adult females were maintained on a 30% sucrose diet and were starved for 12 hours before feeding.

### Data Representation and Statistics

Results obtained in at least 2 independent experiments are shown per individual sand fly/mouse, with a representation of the group mean value±standard deviation. Statistical analysis was performed using GraphPad Prism software v6.01. The nonparametric Mann-Whitney test was used to access statistical differences, with at least P≤0.05.

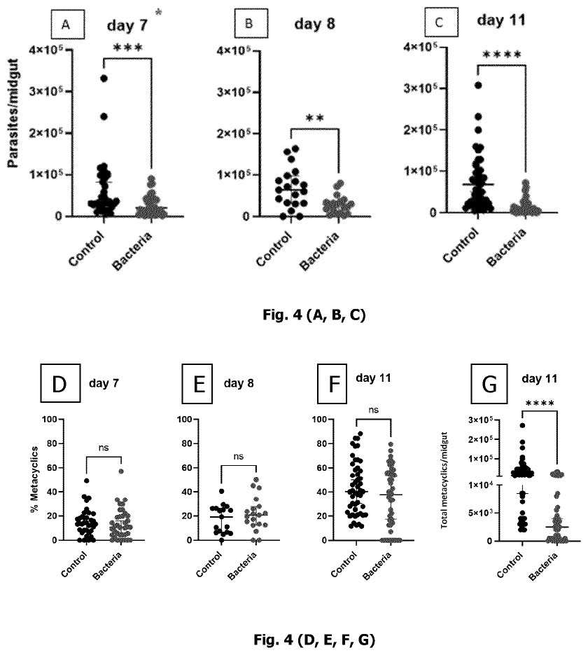

FIG. 4: Impact of Establishment of Leishmania major Parasites in Sandflies Fed with Delftia tsuruhatensis TC1 Bacteria

P. duboscqi sand flies, 5-7 days old, were allowed to feed on cotton rolls impregnated with a suspension of Delftia tsuruhatensis TC1 bacteria (1×10⁸ CFU/ml in 5% sucrose solution) for 24 h (represented as “Bacteria” in the figure); sand flies in the control group (represented as “Control” in the figure) were given 5% sucrose alone.

After an overnight starving period, sand flies were infected by artificial feeding through a chick membrane on defibrinated rabbit blood (Spring Valley Laboratories, MD, USA) containing L. major promastigotes (5×10⁶/ml), as described by Cecílio P, Oristian J, Meneses C. et al. Engineering a vector-based pan-Leishmania vaccine for humans: proof of principle. Scientific Reports. 2020. 10:18653. https://doi.org/10.1038/s41598-020-75410-0.

After infection, blood-fed females were sorted and kept on a 30% sucrose diet. At 7 (and/or 8), and 11 days post infection sand flies were collected to assess the infection status. Briefly, under a stereomicroscope, sand fly midguts were dissected in PBS and transferred to individual microtubes (Denville Scientific) with 50 μL of formalin solution (0.005% in PBS). Midguts were homogenized and 10 μL loaded onto disposable Neubauer chambers (Incyto). Slides were observed under a phase contrast microscope (Zeiss) at 400× magnification.

The total parasite numbers, and the metacyclic frequency [Cecílio P, Pires ACAM, Valenzuela J G, et al. Exploring Lutzomyia longipalpis sand fly vector competence for Leishmania major parasites, The Journal of Infectious Diseases. 2020. 222(7):1199-1203. https://doi.org/10.1093/infdis/jiaa203.] were determined.

Data were represented in the form of dot plots where each symbol corresponds to a single midgut. Statistical differences were assessed using the nonparametric Mann-Whitney test; a value of at least P≤0.05 indicated statistical relevance.

The data illustrated in FIG. 4 (A, B & C) shows that exposure of sandflies to Delftia tsuruhatensis TC1 impacts the establishment of Leishmania parasites within the sandfly midgut. It can be seen that the parasite levels are significantly reduced for sand flies exposed to the bacteria as compared to sandflies without Delftia tsuruhatensis TC1 bacteria. Leishmania infection in sandflies exposed to Delftia tsuruhatensis TC1 showed significant reduction (p<0.0001) (FIG. 4C) in parasite load compared to Delftia tsuruhatensis TC1-treated sand flies at day 7 (p<0.5) (FIG. 4A) or day 8 (p<0.01) (FIG. 4B) post-Leishmania infection.

The data illustrated in FIG. 4 (D, E, F & G) shows that exposure of sandflies to Delftia tsuruhatensis TC1 does not affect the percentage of metacyclic parasites, but that the total number of parasites is significantly reduced within the sandfly midgut for sand flies exposed to the bacteria as compared to sandflies without Delftia tsuruhatensis TC1 bacteria.

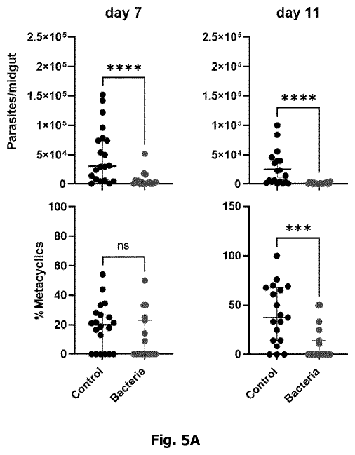

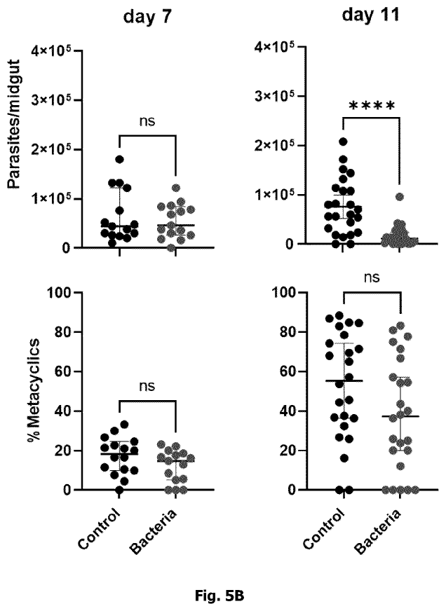

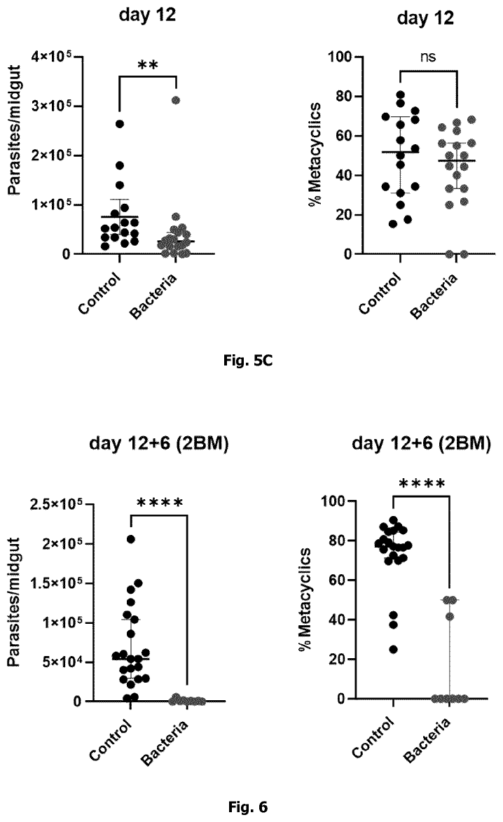

FIG. 5: Impact of Timing of (i) Feed and (ii) Exposure to Delftia tsuruhatensis TC1 Bacteria, on Establishment of Leishmania major Parasites in Sandflies

P. duboscqi sand flies, 5-7 days old, were allowed to feed on cotton rolls impregnated with a suspension of TC1 bacteria (1×10⁸ CFU/ml in 5% sucrose solution) for 24 h; sand flies in the control group were given 5% sucrose alone.

Sand flies were kept on a 30% sucrose diet ad libitum for 6 days, and then, after an overnight starving period (7 days post bacterial feeding), sand flies were infected by artificial feeding through a chick membrane on defibrinated rabbit blood (Spring Valley Laboratories, MD, USA) containing L. major promastigotes (5×10⁶/ml), as previously described.

After infection, blood-fed females were sorted and kept on a 30% sucrose diet. At 7- and 11-days post infection sand flies were collected to assess the infection status as previously described.

The data illustrated in FIG. 5A shows that exposure of sandflies to Delftia tsuruhatensis TC1 impacts the establishment of Leishmania parasites within the sandfly midgut, even where the exposure to bacteria occurs one week prior to infection.

Overnight-starved P. duboscqi sand flies, 5-7 days old, were infected by artificial feeding through a chick membrane on defibrinated rabbit blood (Spring Valley Laboratories, MD, USA) containing L. major promastigotes (5×10⁶/ml). After infection, blood-fed females were sorted and kept on a 30% sucrose diet. Five days post-infection, when all the blood remnants were defecated, sand flies were allowed to feed on cotton rolls impregnated with a suspension of TC1 bacteria (1×10⁸ CFU/ml in 5% sucrose solution) for 24 h; sand flies in the control group were given 5% sucrose alone. Sand flies were then kept on a 30% sucrose diet ad libitum until the termination of the experiment. At 7-, and 11-days post infection sand flies were collected to assess the infection status as described previously.

The data shown in FIG. 5B shows that exposure of previously infected sandflies to Delftia tsuruhatensis TC1 impacts the establishment of Leishmania parasites.

Overnight-starved P. duboscqi sand flies, 5-7 days old, were infected by artificial feeding through a chick membrane on defibrinated rabbit blood (Spring Valley Laboratories, MD, USA) containing L. major promastigotes (5×10⁶/ml). After infection, blood-fed females were sorted and kept on a 30% sucrose diet. Eight days post-infection, after infection started to mature, sand flies were allowed to feed on cotton rolls impregnated with a suspension of TC1 bacteria (1×10⁸ CFU/ml in 5% sucrose solution) for 24 h; sand flies in the control group were given 5% sucrose alone. Sand flies were then kept on a 30% sucrose diet ad libitum until the termination of the experiment. At 12-days post infection sand flies were collected to assess the infection status as described previously.

The data shown in FIG. 5C shows that exposure of sandflies carrying mature infections to Delftia tsuruhatensis TC1 impacts the establishment of Leishmania parasites.

FIG. 6: Impact of Second Bloodmeal on Leishmania major Parasites in Sandflies Fed with Delftia tsuruhatensis TC1 Bacteria

P. duboscqi sand flies, 5-7 days old, were allowed to feed on cotton rolls impregnated with a suspension of TC1 bacteria (1×10⁸ CFU/ml in 5% sucrose solution) for 24 h; sand flies in the control group were given 5% sucrose alone. After an overnight starving period, sand flies were infected by artificial feeding through a chick membrane on defibrinated rabbit blood (Spring Valley Laboratories, MD, USA) containing L. major promastigotes (5×10⁶/ml), as previously described.

After infection, blood-fed females were sorted and kept on a 30% sucrose diet. Twelve days post-infection, sand flies were allowed to take a second bloodmeal. Briefly, sand flies were allowed to blood engorge on an anesthetized naïve mouse for one hour, and the blood-fed females were sorted and kept on a 30% sucrose diet until the termination of the experiment. Six days after the second, non-infected bloodmeal sand flies were collected to assess the infection status as previously described.

The data shown in FIG. 6 shows that infection rates in the TC1-colonized sandflies surviving after a second bloodmeal remain significantly lower than untreated controls thereby showing that a second blood meal does not reverse the infection phenotype.

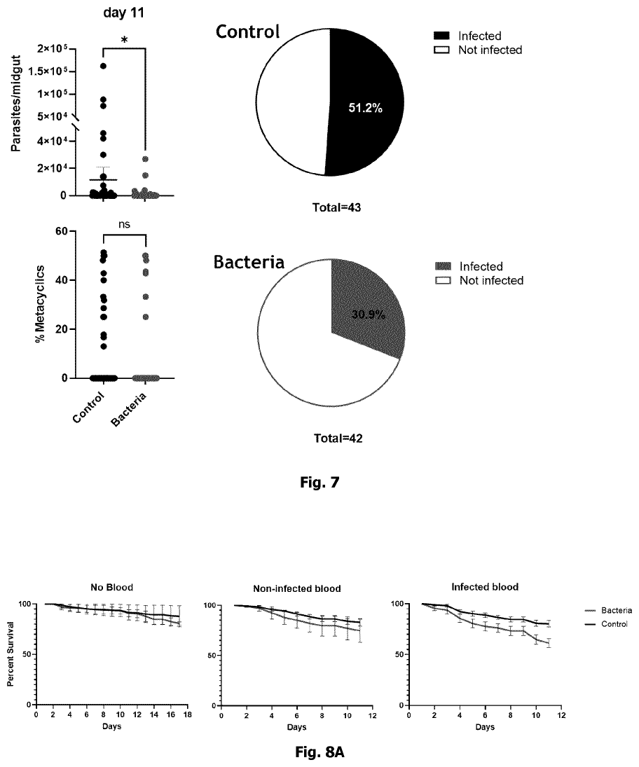

FIG. 7: Impact of Infection Via Bites to Infected Cutaneous Lesions in Mice on Leishmania major Parasites in Sandflies Fed with Delftia tsuruhatensis TC1 Bacteria

BALB/c mice were infected intradermally in the ear pinnae with 1000 L. major metacyclic parasites and kept with water and food ad libitum until the development of typical Cutaneous Leishmaniasis lesions. Then sandflies were allowed to take a bloodmeal from the ears with active lesions using vials with a meshed surface held in place by custom-made clamps. Blood-engorged sandflies were separated into 2 groups. One group was then allowed to feed on the bacteria via sugar-meal overnight (1×10⁸/ml in 5% sterile sucrose solution), while the other received 5% sterile sucrose solution alone. Sandflies from both groups were dissected 11 days post-infection to evaluate their infection status as previously described.

The data shown in FIG. 7 shows that TC1-colonised sandflies show a decreased midgut parasite burden and a lower prevalence of infection when infected naturally via bites on infected cutaneous lesions in mice.

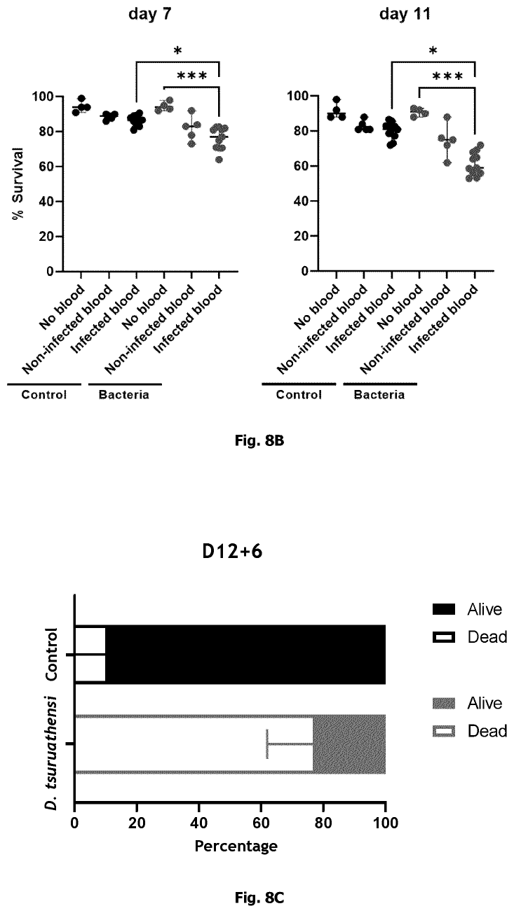

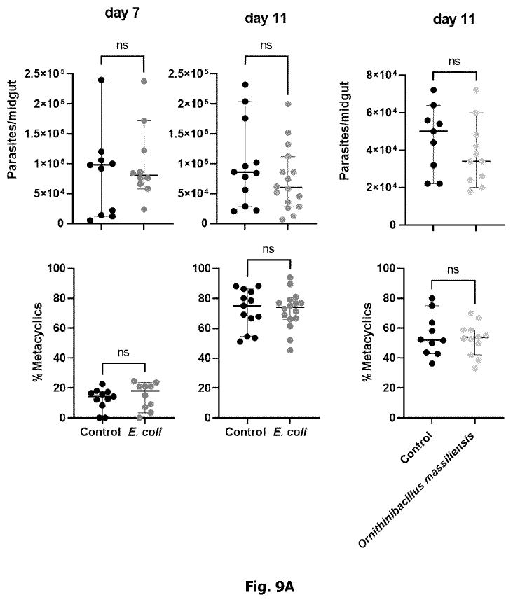

FIG. 8: Sandfly Mortality on Exposure to Delftia tsuruhatensis TC1 Bacteria

P. duboscqi sand flies, 5-7 days old, were allowed to feed on cotton rolls impregnated with a suspension of TC1 bacteria (1×10⁸ CFU/ml in 5% sucrose solution) for 24 h; sand flies in the control group were given 5% sucrose alone.

Bacteria-fed or control sandflies, not fed on blood, artificially fed on non-infected blood, or artificially fed on blood containing L. major promastigotes (5×10⁶/ml) were placed into cardboard pints (100 flies per pint) and kept on a 30% sucrose diet for 11-18 days.

Sandfly mortality was recorded daily, and the dead sandflies were removed also daily. Additionally, for the infected sandflies only, at day 12 post-infection, a second bloodmeal was given (as above described) and the mortality was also recorded daily only considering the sorted blood fed sandflies in the following 6 days. Data are represented in the form of Kaplan-Meier curves, and as dot plots per individual time-point where each symbol refers to a group of 100 sandflies (one bloodmeal), or as parts of a whole (second bloodmeal data). Statistical differences were assessed using the nonparametric Kruskal-Wallis test with post-hoc analysis; a value of at least P≤0.05 was considered statistically relevant.

The data is shown in FIGS. 8A-C. Exposure to the TC1-bacteria slightly increases sandfly mortality but significantly impact the survival of infected sandflies. Mortality increases significantly after the second bloodmeal, only in TC1-colonized sandflies infected with L. major parasites.

It can be seen from the data that TC1 bacteria not only decreases the infection burden in sandflies but also reduces the overall number of infected sandflies.

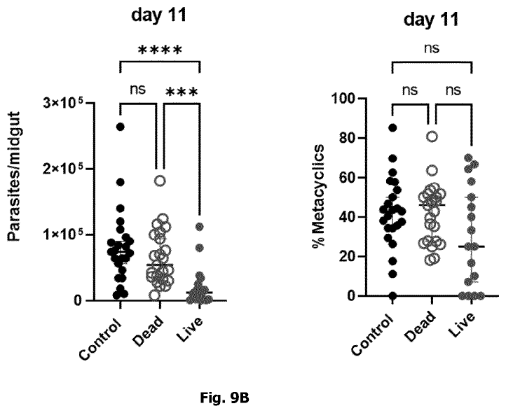

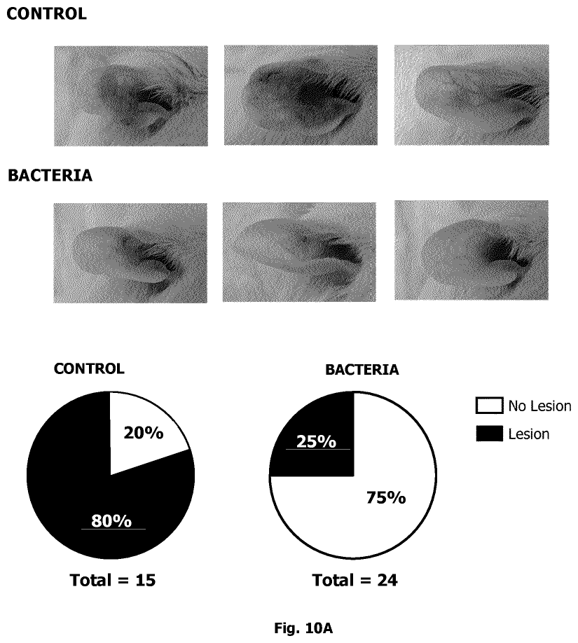

FIG. 9: Experiment to Establish if the Reduction in Burden of Leishmania major Parasites in Sandflies is Specific to Live Delftia TC1 (a Gram Negative Bacteria) or is Applicable to all Gram Negative Bacteria

P. duboscqi sand flies, 5-7 days old, were allowed to feed on cotton rolls impregnated with a suspension of gram negative ampicillin-resistant E. coli, or an Ornithinibacillus massiliensis strain isolated from the microbiota of healthy P. duboscqi sand flies from our colony (1×10⁸ CFU/ml in 5% sucrose solution) for 24 h; sand flies in the control group were given 5% sucrose alone. After an overnight starving period, sand flies were infected by artificial feeding through a chick membrane on defibrinated rabbit blood (Spring Valley Laboratories, MD, USA) containing L. major promastigotes (5×10⁶/ml), as previously described. After infection, blood-fed females were sorted and kept on a 30% sucrose diet.

At (7-, if applicable, and) 11-days post infection sand flies were collected to assess the infection status as described previously. The data shown in FIG. 9A illustrates that E. coli and Ornithinibacillus massiliensis do not have the same effect on parasitic infection in the sandfly midgut as Delftia tsuruhatensis. The effects seen for TC1 are not universal for gram-negative bacterial infection but are specific to TC1.

Live TC1 bacteria were heat-inactivated at 95° C. for 10 minutes to produce dead TC1 bacteria. P. duboscqi sand flies, 5-7 days old, were allowed to feed on cotton rolls impregnated with a suspension of live or dead TC1 bacteria (1×10⁸ CFU/ml in 5% sucrose solution) for 24 h; sand flies in the control group were given 5% sucrose alone. After an overnight starving period, sand flies were infected by artificial feeding through a chick membrane on defibrinated rabbit blood (Spring Valley Laboratories, MD, USA) containing L. major promastigotes (5×10⁶/ml), as previously described. After infection, blood-fed females were sorted and kept on a 30% sucrose diet.

At 11 days post infection sand flies were collected to assess the infection status as described previously. The data shown in FIG. 9B illustrates that dead Delftia tsuruhatensis does not have the same effect on parasitic infection in the sandfly midgut as live Delftia tsuruhatensis.

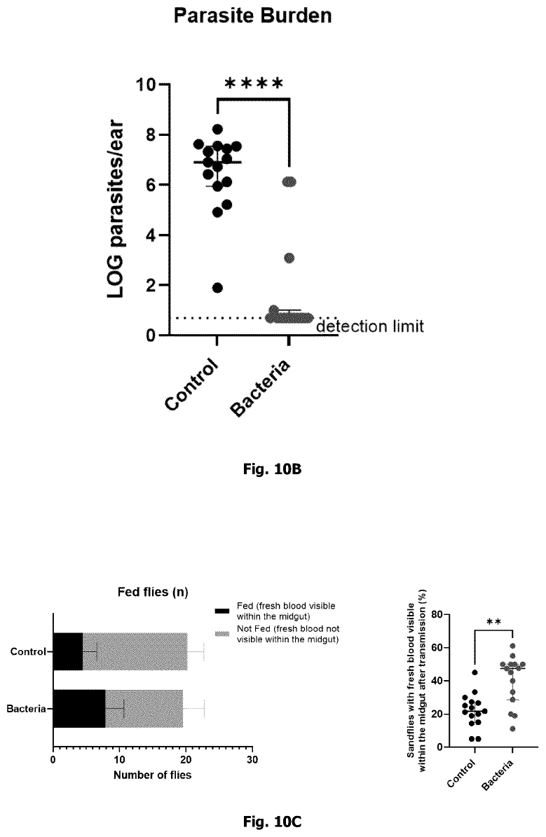

FIG. 10 Illustrates the Effect of Delftia tsuruhatensis on Interrupting Transmission of Leishmania Disease to Naïve Mice.

P. duboscqi sand flies, 5-7 days old, were allowed to feed on cotton rolls impregnated with a suspension of TC1 bacteria (1×10⁸ CFU/ml in 5% sucrose solution) for 24 h; sand flies in the control group were given 5% sucrose alone. After an overnight starving period, sand flies were infected by artificial feeding through a chick membrane on defibrinated rabbit blood (Spring Valley Laboratories, MD, USA) containing L. major promastigotes (5×10⁶/ml), as previously described.

After infection, blood-fed females were sorted and kept on a 30% sucrose diet, ad libitum. Eleven days post-infection, 20 sand flies were applied each ear of BALB/c mice, using vials with a meshed surface held in place by custom-made clamps, and allowed to feed for at least 30 minutes. The number of blood-fed flies was determined by observing them under a stereomicroscope. Thereafter, mice were monitored on a weekly basis to follow the development of lesions caused by L. major infection.

Images of individual ears were captured weekly using a smartphone (FIG. 10A). Animals were euthanized 4 weeks post infection for parasite burden determination via the limiting dilution method, as reported in Dey R, Joshi A B, Oliveira F, et al. Gut microbes egested during bites of infected sand flies augment severity of leishmaniasis via inflammasome-derived IL-1β. Cell Host Microbe. 2018. 23:134-43.e6. https://doi.org/10.1016/j.chom.2017.12.002. Data is shown in FIG. 10B. Data were represented in the form of dot plots where each symbol corresponds to a single mouse ear. Statistical differences were assessed using the nonparametric Mann-Whitney test; a value of at least P≤0.05 indicated statistical relevance.

FIG. 10A: Mice which were bitten by leishmania-infected sandflies previously exposed to Delftia tsuruhatensis showed an absence of lesions in the ear, caused by L. major infection compared to mice bitten by infected sandflies not exposed to Delftia tsuruhatensis (images in TEST panel).

FIG. 10B: Parasite burden in mice ear's bitten by leishmania-infected sandflies previously exposed to Delftia tsuruhatensis (bacteria) was significantly reduced compared to mice bitten by infected sandflies not exposed to Delftia tsuruhatensis (control).

FIG. 10C: Sandflies “colonized” with the TC1 Bacteria were more capable of taking a second bloodmeal because the reduced infection caused by TC1 leads to reduction in the blockage of the gut.

The data show that the Delftia TC1 bacteria impacts the development of Leishmania major parasites in P. duboscqi sandflies, as seen by the significantly lower number of both total parasites and the infectious metacyclic forms (90% reduction) in bacteria-fed versus control sandflies. This phenotype was consistently observed, regardless of the timing of bacterial feeding (one week prior to infection Vs. one day prior to infection Vs. five days after infection), and even potentiated in the context of sandflies given a second, uninfected, bloodmeal. Moreover, the TC1 bacteria not only decreased the infection burden in sandflies, but also the overall number of infected sandflies, leading to an increase in the mortality of Leishmania-infected sandflies, but not of sugar-fed or blood-fed (uninfected bloodmeal) sandflies. The data also show that Leishmania-infected, TC1 bacteria-fed sandflies are less able to transmit Leishmania major parasites and cause disease in the context of a mouse model of cutaneous leishmaniasis (active lesions observed in 25% of animals bitten by bacteria-fed flies versus 80% in the control group; parasites were detected in 27% of animals bitten by bacteria-fed flies versus 100% of animals in the control group).

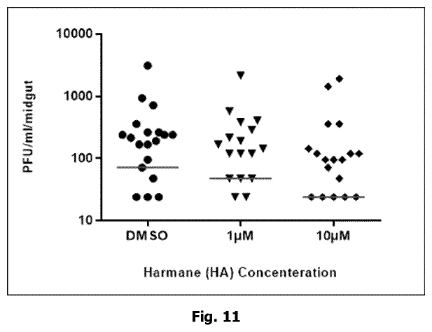

FIG. 11: Effect of Harmane in Interruption of Zika Virus Replication in Aedes aegypti Mosquitoes

3-to-5-day-old Aedes aegypti strain SBE mosquitoes were fed via membrane feeders on a blood meal (rabbit blood) containing approximately 10⁷ PFU/ml Zika virus and the indicated concentrations of harmane dissolved in dimethyl sulphoxide (DMSO). The bloodmeal fed to the control group contained no harmane. Fully engorged mosquitoes were selected and kept on a 10% sucrose diet. In total, 30 mosquitoes were analyzed per treatment.

After 7 days, the abdomens of mosquitoes were dissected and placed in 2 mL tubes containing glass beads and 300 μL of DMEM medium. Samples were homogenized. A 1:10 dilution series of this solution was dispersed onto 96-well plates containing VERO cells. 5-6 days later, the medium was discarded and viral plaques were fixed and developed with staining reagent (1% crystal violet in 1,1 methanol/acetone solution) at room temperature for 1 h. 5

Plates were rinsed with distilled water and air-dried. Plaques were counted and multiplied by the corresponding dilution factors to calculate the number of plaque-forming units (PFUs). The number of PFUs is shown in FIG. 11. Horizontal red lines are median values. A dose dependent decrease of PFU/mL was observed in the Harmane treated samples compared to the control group. Statistical differences were assessed using the nonparametric Mann-Whitney test. No significant differences were observed in the treated groups compared to the DMSO-control.

The data in FIG. 11 shows a reduced level of Zika virus in mosquitoes which have ingested harmane.

What is claimed is:

1. A method of reducing transmission of a vector borne disease, wherein the method comprises the step of bringing at least one vector into contact with a composition comprising pure bacteria of the Delftia genus and/or 1-Methyl-9H-pyrido[3,4-b]indole or a pharmaceutically acceptable salt thereof,
wherein the vector borne disease is selected from Human African Trypanosomiasis, Anaplasmosis, Babesiosis, Borrelia miyamotoi disease, Bourbon virus, Borrelia mayonii disease, Chikungunya virus, Chagas disease, Dirofilariasis, Eastern Equine Encephalitis, Ehrlichia muris-like disease, Ehrlichiosis, Filariasis, Heartland virus, Japanese encephalitis, Leishmaniasis, Lyme Disease, Mayaro virus disease, Pacific Coast tick fever, Rocky Mountain spotted fever, St. Louis encephalitis, Tularemia, Western equine encephalitis, West Nile virus, Yellow fever, and Zika virus.

2. A method of reducing transmission of a parasite, wherein the method comprises the step of bringing at least one vector into contact with a composition comprising pure bacteria of the Delftia genus and/or 1-Methyl-9H-pyrido[3,4-b]indole or a pharmaceutically acceptable salt thereof,
wherein the parasitic disease is selected from Human African Trypanosomiasis, Anaplasmosis, Babesiosis, Borrelia miyamotoi disease, Borrelia mayonii disease, Chagas disease, Cryptosporidium, Dirofilariasis, Ehrlichia muris-like disease, Ehrlichiosis, Filariasis, Leishmaniasis, Lyme Disease, Pacific Coast tick fever, Rocky Mountain spotted fever, and Tularemia.

3. The method according to claim 1, wherein the bacteria is Delftia tsuruhatensis and/or wherein the composition further comprises at least one carrier, delivery vehicle, adjuvant, solvent, stabilizer, or preservative.

4. The method according to claim 1, wherein the composition comprises an attractant.

5. The method according to claim 4, wherein the attractant is a sugar source or pheromone.

6. The method according to claim 1, wherein the method reduces: (i) Zika virus, and/or (ii) Zika virus transmission.

7. The method according to claim 6, wherein the method reduces: (i) Zika virus, and/or (ii) Zika virus transmission in a mosquito.

8-15. (canceled)

16. The method according to claim 7, wherein the mosquito is a mosquito of the Aedes genus.

17. The method according to claim 16, wherein the mosquito is Aedes albopictus, or Aedes aegypti.

18-23. (canceled)

24. The method according to claim 1, wherein the method reduces: (i) Leishmaniasis, and/or (ii) Leishmaniasis transmission.

25. The method according to claim 24, wherein the method reduces: (i) Leishmaniasis, and/or (ii) Leishmaniasis transmission in a Sand fly,

26. The method according to claim 25, wherein the Sand fly is of the genus Phlebotomus or Lutzomyia.

27-29. (canceled)

30. A composition comprising pure bacteria of the Delftia genus and 1-Methyl-9H-pyrido[3,4-b]indole or a pharmaceutically acceptable salt thereof.

31. A composition of claim 30 further comprising at least one attractant, carrier, delivery vehicle, adjuvant, solvent, stabilizer, or preservative.

32. A composition of claim 31 comprising an attractant.

33. A composition of claim 32, wherein the attractant is a sugar source or pheromone.
